(19) United States
(12) Patent Application Publication
(10) Pub. No.: US 2025/0213345 A1
(43) Pub. Date: Jul. 3, 2025

(54) STENT WITH FEATURES TO REDUCE FOOD IMPACTION

(71) Applicant: BOSTON SCIENTIFIC SCIMED, INC., Maple Grove, MN (US)

(72) Inventors: ROBB MORSE GAVALIS, Westborough, MA (US); PETER L. DAYTON, Brookline, MA (US)

(73) Assignee: BOSTON SCIENTIFIC SCIMED, INC., Maple Grove, MN (US)

(21) Appl. No.: 18/985,732

(22) Filed: Dec. 18, 2024

Related U.S. Application Data
(60) Provisional application No. 63/612,141, filed on Dec. 19, 2023.

(51) Int. Cl. A61F 2/04; A61F 2/00

(52) U.S. Cl. CPC A61F 2/04; A61F 2002/0081; A61F 2002/045; A61F 2230/0006; A61F 2230/0069; A61F 2230/0071

(57) ABSTRACT

Stents and/or implants for reducing food impaction. An illustrative transluminal implant, may comprise an elongated tubular body extending from a first end to a second end. The elongated tubular body may comprise a scaffolding forming a plurality of cells and defining a lumen extending from the first end to the second end of the elongated tubular body and at least one strut bisecting a luminal opening of the scaffolding to define two or more openings each having a cross-sectional dimension less than a cross-sectional dimension of the luminal opening.

### CROSS REFERENCE TO RELATED APPLICATIONS

This application claims the benefit of and priority to U.S. Provisional Patent Application Ser. No. 63/612,141, filed on Dec. 19, 2023, the disclosure of which is incorporated herein by reference.

### TECHNICAL FIELD

The present disclosure relates generally to methods and apparatuses for various ailments. More particularly, the disclosure relates to different configurations and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain patency of the structure or to maintain patency between two structures that have been combined via a stent for an alternate drainage path (e.g., transmural drainage). These devices are manufactured by any one of a variety of different manufacturing methods and may be used according to any one of a variety of methods for a variety of applications. Of the known medical stents, delivery systems, and methods, each has certain advantages and disadvantages. For example, some stents may become occluded. Occlusive events can be a combination of local factors, such as, but not limited to inadequate stricture resolution and irregular bile properties and/or related to stent design. In some examples, occlusive events may occur due to the location of the stent within the body. For example, a stent positioned with an end thereof in the stomach may experience food impaction or food migration into the lumen of the stent. Thus, there is an ongoing need to provide alternative stent designs which reduce or alleviate the risk of stent occlusion from food impaction.

### SUMMARY

The disclosure is directed to several alternative designs, materials and methods of manufacturing medical device structures and assemblies, and the use thereof. An example medical device may include a stent.

In a first example, a transluminal implant may comprise an elongated tubular body extending from a first end to a second end. The elongated tubular body may comprise a scaffolding forming a plurality of cells and defining a lumen extending from the first end to the second end of the elongated tubular body and at least one strut bisecting a luminal opening of the scaffolding to define two or more openings each having a cross-sectional dimension less than a cross-sectional dimension of the luminal opening.

Alternatively or additionally to any of the examples above, in another example, the transluminal implant may further comprise a proximal cage disposed adjacent to the first end.

Alternatively or additionally to any of the examples above, in another example, the at least one strut may form a portion of the proximal cage.

Alternatively or additionally to any of the examples above, in another example, the proximal cage may have a generally oblate spheroid shape.

Alternatively or additionally to any of the examples above, in another example, the proximal cage may have a generally hemi-spherical shape.

Alternatively or additionally to any of the examples above, in another example, the proximal cage may have an outer diameter greater than an outer diameter of the second end of the elongated tubular body.

Alternatively or additionally to any of the examples above, in another example, the at least one strut may form a plurality of loops.

Alternatively or additionally to any of the examples above, in another example, a loop of the plurality of loops may at least partially overlap a preceding loop of the plurality of loops.

Alternatively or additionally to any of the examples above, in another example, the plurality of loops may extend about an entirety of a circumference of the luminal opening.

Alternatively or additionally to any of the examples above, in another example, the plurality of loops may be woven.

Alternatively or additionally to any of the examples above, in another example, the at least one strut may intersect with another strut across the luminal opening.

Alternatively or additionally to any of the examples above, in another example, the transluminal implant may further comprise a coating disposed over at least some of the plurality of cells.

Alternatively or additionally to any of the examples above, in another example, the two or more openings may be free from the coating.

Alternatively or additionally to any of the examples above, in another example, the coating may be disposed over at least some of the two or more openings.

Alternatively or additionally to any of the examples above, in another example, the second end of the elongated tubular body may include a flared distal end region.

In another example, a transluminal implant may comprise an elongated tubular body extending from a first end to a second end. The elongated tubular body may comprise a scaffolding forming a plurality of cells and defining a lumen extending from the first end to the second end of the elongated tubular body, a proximal cage disposed adjacent to the first end of the elongated tubular body, and at least one strut forming a proximal portion of the proximal cage and bisecting a luminal opening of the scaffolding to define two or more openings each having a cross-sectional dimension less than a cross-sectional dimension of the luminal opening.

Alternatively or additionally to any of the examples above, in another example, the proximal cage may comprise a distal shoulder, a proximal end, and a curved side wall extending between the distal shoulder and the proximal end.

Alternatively or additionally to any of the examples above, in another example, the distal shoulder may extend radially from an intermediate region of the elongated tubular body.

Alternatively or additionally to any of the examples above, in another example, the proximal cage may have a generally oblate spheroid shape.

Alternatively or additionally to any of the examples above, in another example, the proximal cage may have a generally hemi-spherical shape.

Alternatively or additionally to any of the examples above, in another example, the proximal cage may have an outer diameter greater than an outer diameter of the second end of the elongated tubular body.

Alternatively or additionally to any of the examples above, in another example, the at least one strut may form a plurality of loops.

Alternatively or additionally to any of the examples above, in another example, a loop of the plurality of loops may at least partially overlap a preceding loop of the plurality of loops.

Alternatively or additionally to any of the examples above, in another example, the plurality of loops may extend about an entirety of a circumference of the luminal opening.

Alternatively or additionally to any of the examples above, in another example, the at least one strut may intersect with another strut across the luminal opening.

Alternatively or additionally to any of the examples above, in another example, the transluminal implant may further comprise a coating disposed over at least some of the plurality of cells.

Alternatively or additionally to any of the examples above, in another example, the two or more openings may be free from the coating.

Alternatively or additionally to any of the examples above, in another example, the coating may be disposed over at least some of the two or more openings.

In another example, a transluminal implant may comprise an elongated tubular body extending from a proximal end to a distal end. The elongated tubular body may comprise a scaffolding forming a plurality of cells and defining a lumen extending from the proximal end to the distal end of the elongated tubular body, a proximal cage disposed adjacent to the proximal end of the elongated tubular body, the proximal cage including a mesh bisecting a luminal opening of the scaffolding to define a plurality of openings each having a cross-sectional dimension less than a cross-sectional dimension of the luminal opening, and a coating disposed over a portion of the scaffolding.

Alternatively or additionally to any of the examples above, in another example, at least some of the plurality of openings may be free from the coating.

Alternatively or additionally to any of the examples above, in another example, a distal end region of the elongated tubular body may be free from the coating.

Alternatively or additionally to any of the examples above, in another example, the mesh may comprise one or more intersecting struts.

Alternatively or additionally to any of the examples above, in another example, the mesh may comprise a plurality of overlapping loops.

In another example, a transluminal implant may comprise an elongated tubular body extending from a first end to a second end. The elongated tubular body may comprise a scaffolding forming a plurality of cells and defining a lumen extending from the first end to the second end of the elongated tubular body, a coating disposed over at least some of the plurality of cells, and a flexible sheath extending from a proximal end to a distal end and defining a lumen extending therethrough, the proximal end extending proximally beyond the first end of the elongated tubular body.

Alternatively or additionally to any of the examples above, in another example, an inner diameter of the flexible sheath may reduce towards the proximal end of the flexible sheath.

Alternatively or additionally to any of the examples above, in another example, the flexible sheath may be secured to an outer surface of the coating.

Alternatively or additionally to any of the examples above, in another example, the flexible sheath may be secured to an inner surface of the coating.

Alternatively or additionally to any of the examples above, in another example, the flexible sheath may be formed as a single monolithic structure with the coating.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cross-sectional view of an illustrative stent implanted in a body to create a pathway between an intrahepatic (bile) duct and the stomach;

FIG. 2 illustrates a side view of an illustrative implant that is adapted for use in fluidly coupling two different body lumens in an expanded state;

FIG. 3 illustrates a proximal end view of the illustrative stent of FIG. 2;

FIG. 4 illustrates a side view of another illustrative implant in an expanded state;

FIG. 5 illustrates a side view of the illustrative stent of FIG. 4 having an alternative coating arrangement;

FIG. 6 illustrates a side view of another illustrative implant, such as, but not limited to, a stent, in an expanded state;

FIG. 7 illustrates a cross-sectional view of the illustrative stent of FIG. 6; and

FIG. 8 illustrates is a cross-sectional view of the illustrative stent of FIG. 6 having an alternative placement of the flexible sleeve.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

In some instances, it may be desirable to provide an endoluminal implant, or stent, that can deliver luminal patency within the pancreaticobiliary tree of a patient. The relatively narrow biliary tract ducts consist of a series of bifurcations linking the liver, gallbladder, and pancreas via the papilla to the duodenal space for the transportation of bile and related enzymic substances for many metabolic functions but most commonly the body's ability to digest and absorb fats and vitamins D and K. However, blockages may occur in the pancreaticobiliary tree due to tumor related duct narrowing, stricture formation, infections, or stone and sludge generation among other etiologies. Endoscopic retrograde cholangiopancreatography (ERCP) is used to diagnose and treat these duct narrowings, regardless of the malignant or benign nature of the disease. However, ERCP may not always be an option or may be unsuccessful in difficult anatomies or challenging disease states. Hepaticogastrostomy (HGS) may be used in cases where ERCP is not an option or fails. Other procedures, such as, but not limited to, choledochoduodenostomy (CDS) may be used to directly connect the common bile duct to the duodenum.

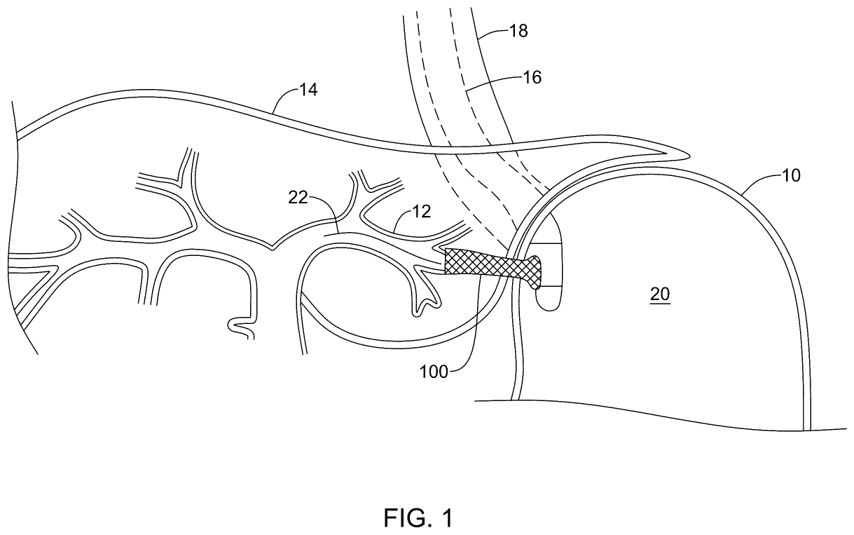

HGS is a procedure that targets an intrahepatic duct directly from the stomach and places a stent creating an artificial pathway (bridging the peritoneal cavity) to facilitate ongoing internal biliary drainage. FIG. 1 is a partial cross-sectional view of an illustrative stent 100 implanted in a body to create a pathway between an intrahepatic (bile) duct 12 and the stomach 10. The HGS procedure is completed using an endoscopic ultrasound scope or other endoscope 16 advanced through the esophagus 18 that allows the endoscopist to “see” through the wall of the stomach 10 and into the liver 14 prior to gaining access. The endoscopist may position the scope 16 with an appropriate clear trajectory to the intrahepatic duct 12. Guidewire access to the duct 12 is then made using a needle followed by enlargement of the tract using a balloon and/or cystotome. Alternatively, access is gained using a specialized all in one device. The guidewire 22 is advanced from the stomach 10 into the duct 12. Finally, a stent 100 is advanced and deployed to create and maintain the bridge between the duct 12 and an interior 20 of the stomach 10 thus facilitating ongoing internal biliary drainage. An additional factor endoscopists balance when positioning the endoscope 16 for this procedure is also the proximal positioning of the stent 100 within the stomach to minimize food impaction/migration into the stent lumen. The present disclosure is directed towards alternative stent designs which reduce and/or alleviate the risk of stent occlusion from food impaction. While the present disclosure is described with respect to an HGS procedure, the devices, systems, and/or methods described herein may be used in stents, endoluminal implants, or transluminal implants where the proximal end terminates in the upper GI tract. This may include devices placed during a CDS procedure or during a successful ERCP procedure, among others. Further, while the present disclosure is described with respect to the pancreaticobiliary ductal system, the devices, systems, and/or methods described herein may be used in stents, endoluminal implants, or transluminal implants positioned in other parts of the body, such as, but not limited to, bodily tissue, bodily organs, vascular lumens, non-vascular lumens and combinations thereof, such as, but not limited to, in the coronary or peripheral vasculature, trachea, bronchi, colon, small intestine, esophagus, biliary tract, urinary tract, prostate, brain, stomach, and the like.

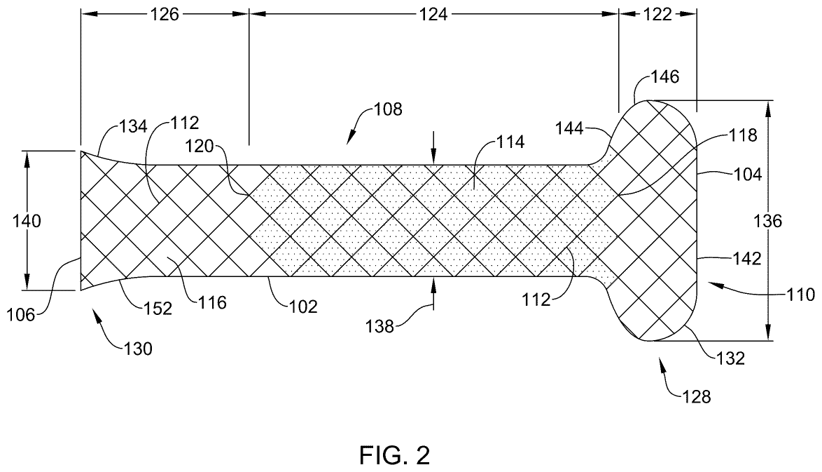

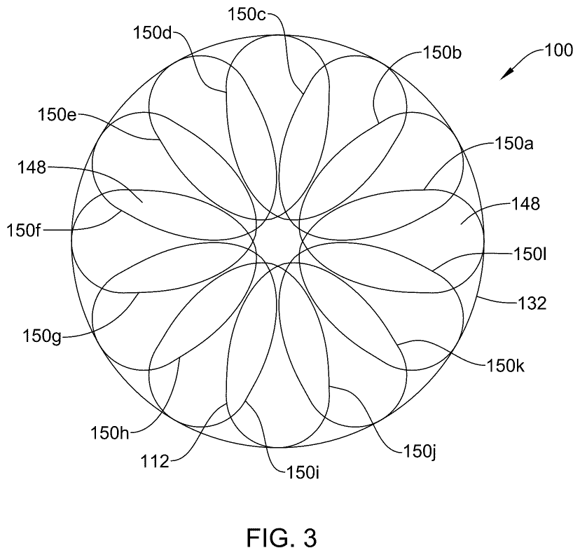

FIG. 2 illustrates a side view of an illustrative implant 100 that is adapted for use in fluidly coupling two different body lumens, such as, but not limited to, a stent, in an expanded state. FIG. 3 illustrates a proximal end view of the illustrative stent 100 of FIG. 2. In some instances, the stent 100 may be formed from an elongated tubular member 102. While the stent 100 is described as generally tubular, it is contemplated that the stent 100 may take any cross-sectional shape desired. The stent 100 may have a first, or proximal, end 104, a second, or distal, end 106, and an intermediate region 108 disposed between the first end 104 and the second end 106. The stent 100 may include a lumen 110 extending from a first opening adjacent the first end 104 to a second opening adjacent to the second end 106 to allow for the passage of bile, fluids, and the like therethrough. In some cases, as will be described in more detail herein, the first opening adjacent to the first end 104 may include one or more or a plurality of openings.

The stent 100 may be radially expandable from a first radially collapsed configuration (not explicitly shown) to a second radially expanded configuration, as shown in FIG. 2. The stent 100 may be structured to extend across two non-adherent structures/tissues and to apply a radially outward pressure to create an opening or passage between the two non-adherent structures/tissues, thereby forming an anastomosis between the two separate anatomical structures.

The tubular member 102 of the stent 100 may have a scaffold structure, fabricated from one or more, or a plurality of interwoven filaments or struts 112. The scaffold structure may extend from the first end 104 to the second end 106 of the stent 100. For example, the scaffold structure, and thus the filament(s) thereof, may extend continuously from the first end 104 to the second end 106 of the stent 100. In some embodiments, the stent 100 may be formed with one filament interwoven with itself (e.g., knitted) to form the scaffold structure. In other embodiments, the stent 100 may be formed with several interwoven filaments (e.g., braided) to form the scaffold structure. Thus, in such instances one or more of the filament(s) forming the scaffold structure may extend continuously from the first end 104 to the second end 106 of the stent 100. In still another embodiment, the stent 100 may include a laser cut tubular member to form the scaffold structure. A laser cut tubular member may have an open and/or closed cell geometry including one or more interconnected struts formed as a monolithic structure from the tubular member. In such instances, the laser cut tubular member forming the scaffold structure may extend continuously from the first end 104 to the second end 106 of the stent 100.

In some instances, an inner and/or outer surface of the scaffold structure of the stent 100 may be entirely, substantially or partially, covered with a polymeric covering or layer 114. For example, a covering or coating 114 may extend across the open cells of the scaffold structure to prevent tissue ingrowth into the lumen 110 of the stent 100. In some cases, the covering or coating 114 may prevent food or particulate ingress into the lumen 110 or fluid leakage from the lumen 110 along the coated regions. While not explicitly shown, the covering or coating 114 may include an outer layer disposed over an outer surface of the scaffold structure and/or an inner layer disposed over an inner (e.g., luminal) surface of the scaffold structure. In some embodiments, the stent 100 may include only an outer polymeric covering on an outer surface of the scaffold structure. In other embodiments, the stent 100 may include only an inner polymeric covering on an inner surface of the scaffold structure. In some instances, an inner layer and an outer layer may be formed as a single unitary structure to form the covering or coating 114. In other embodiments, an inner layer and an outer layer may be formed as separate layers to collectively form the covering or coating 114. The inner and outer layers may be formed from the same material or different materials, as desired. The covering or coating 114 may span or be disposed within openings or interstices defined between adjacent stent filaments or struts 112 of the scaffold structure. It can be appreciated that as an inner layer and an outer layer extend outwardly and inwardly, respectively, they may touch and/or form an interface region within the spaces (e.g., openings, cells, interstices) in the wall of the scaffold structure of the stent 100. For example, the inner and outer layers may extend into the openings 116 defined between adjacent stent struts 112 and form an interface region. Further, the inner and outer layers may additionally extend between adjacent filaments or struts 112, thereby filling any space between adjacent filaments or struts 112, and thus prevent tissue ingrowth into the lumen of the stent 100. The covering or coating 114 may extend along an entire length of the stent 100 from the first end 104 to the second end 106. In other embodiments, the covering or coating 114 may extend along only a portion of the stent 100. For example, the covering or coating 114 may extend from a first end 118 distal to the proximal end 104 of the stent 100 to a second end 120 proximal to the distal end 106 of the stent 100 to define a proximal end region 122 free from the covering or coating 114, an intermediate coated region 124, and a distal end region 126 free from the covering or coating 114. It is contemplated that the proximal end region 122 may remain uncoated to allow fluid or bile to pass through the lumen 110 of the stent 100 and exit the lumen 110 at the proximal end region 122 while restricting large particle ingress. In some embodiments, the uncoated distal end region 126 may have a length greater than the uncoated proximal end region 122. However, this is not required. In some examples, the uncoated proximal end region 122 may have a length greater than the uncoated distal end region 126. In yet other examples, the uncoated proximal end region 122 and the uncoated distal end region 126 may have approximately the same length. It is contemplated that the length of the uncoated proximal end region 122, the coated intermediate region 124, and/or the uncoated distal end region 126 may be determined based on the desired application, implant location, etc. In some examples, the stent 100 may be fully coated with openings for fluid flow processed into portions of the coating 114.

It is contemplated that the scaffold structure, e.g., the filaments and/or struts, of the stent 100 can be made from a number of different materials such as, but not limited to, metals, metal alloys, shape memory alloys, and/or polymers, as desired, enabling the stent 100 to be expanded into shape when accurately positioned within the body. In some instances, the material may be selected to enable the stent 100 to be removed with relative ease as well. For example, the stent 100 can be formed from alloys such as, but not limited to, nitinol and Elgiloy®. Depending on the material selected for construction, the stent 100 may be self-expanding or require an external force to radially expand the stent 100. In some embodiments, filaments may be used to make the stent 100, which may be composite filaments, for example, having an outer shell made of nitinol and having a platinum core. It is further contemplated that the filaments of the stent 100 may be formed from polymers including, but not limited to, polyethylene terephthalate (PET).

In some instances, in the radially expanded configuration, the stent 100 may include a first end region 128 proximate to the first end 104 and a second end region 130 proximate to the second end 106. In some embodiments, the first end region 128 may include a proximal cage 132 positioned adjacent to the first end 104 and the second end region 130 may include a distal end region 134 positioned adjacent to the second end 106 of the stent 100. It is contemplated that the proximal cage 132 and/or the distal end region 134 may be formed at a same time as the elongated tubular member 102 of the stent 100. For example, the proximal cage 132 and/or the distal end region 134 may be formed from the one or more, or plurality of filaments or struts forming the scaffold structure that extend continuously from the first end 104 to the second end 106 of the stent 100 as the elongated tubular member 102 is woven, knit, laser cut, or the like. The proximal cage 132 may be configured to engage an interior portion of the wall of the body cavity or body lumen. For example, the proximal cage 132 may be positioned against an interior of a first body lumen (e.g., the wall of the stomach 10). The distal end region 134 may be configured to be positioned within another lumen separate (e.g., duct 12) from the first body lumen. However, the distal end region 134 may increase in diameter relative to the intermediate region 108 to exert a radially outward force on a surface of the lumen of the duct 12 to facilitate anchoring of the stent 100. Thus, the stent 100 may be positioned to traverse between two separate anatomical structures. For example, in FIG. 1 the stent 100 is positioned such that it extends between the stomach 10 and the intrahepatic duct 12. The proximal cage 132 may be positioned in the stomach 10 and the distal end region 134 may be positioned in the intrahepatic duct 12. The intermediate region 108 of the stent 100 extending between the proximal cage 132 and the distal end region 134 may extend through the wall of the stomach 10 and into the lumen of the intrahepatic duct 12, interconnecting the two anatomical structures. In some cases, at least a portion of the proximal cage 132 may contact an interior of the wall of the stomach 10 and/or the distal end region 134 may contact an inner surface of the intrahepatic duct 12. The proximal cage 132 and/or the distal end region 134 may anchor the stent 100 against the stomach wall and the intrahepatic duct 12 so that stent 100 is inhibited or prevented from migrating. However, this is not required.

The proximal cage 132 may have an outer diameter 136 that is greater than an outer diameter 138 of the intermediate region 108. In some examples, the proximal cage 132 may have a generally spheroid shape, such as, but not limited to, a generally oblate spheroid including a proximal end 142, a distal shoulder 144, and a curved side wall 146 extending between the proximal end 142 and the distal shoulder 144. It is contemplated that the proximal cage 132 may take other shapes, such as, but not limited to, spherical, hemi-spherical, rectangular prism, irregular, or the like. The distal shoulder 144 of the proximal cage 132 may extend radially from the intermediate region 108 of the elongated tubular member 102. It is contemplated that the transition from the cross-sectional area of the intermediate region 108 to the proximal cage 132 may be gradual, sloped, or occur in an abrupt step-wise manner, as desired. In some examples, the distal shoulder 144 may extend generally orthogonal to a longitudinal axis of the elongated tubular member 102. However, it is contemplated that the distal shoulder 144 may extend at non-orthogonal angles, as desired. The angle of the distal shoulder 144 may be selected based on the target anatomy. In some cases, the distal shoulder 144 may provide a mechanical stop between the proximal cage 132 and the target anatomy. The curved side wall 146 may have a curved semi-circular shape that gradually increases in cross-sectional dimension and then gradually decreases in cross-sectional dimension in a direction parallel to the longitudinal axis of the elongated tubular body 102. However, the curved side wall 146 may take other shapes including curved, linear, or irregular shapes, as desired.

Referring additionally to FIG. 3, the proximal end 142 of the proximal cage 132 may include more struts (e.g., filaments) 112 of the elongated body 102 extending across the proximal opening of the lumen 110 of the stent 100. Said differently, the struts or filaments 112 may bisect the proximal luminal opening of the stent 100 to form a mesh or screen across the proximal opening of the lumen 110 of the stent 100. The struts or filaments 112 may extend transverse to a longitudinal axis of the stent 100. The one or more struts or filaments 112 may intersect or overlap with one another to define a plurality of openings 148 each having a smaller cross-sectional dimension than the cross-sectional dimension of the proximal luminal opening of the elongated tubular member 102. For purposes of clarity and simplicity, not every opening 148 is labeled. It is contemplated that the density or number of the struts or filaments 112 extending across the proximal opening may be inversely proportional to the size of the openings 148. For example, the denser the struts or filaments 112 (e.g., the greater the number of struts 112 or the greater the number of intersections or overlaps), the smaller the openings 148. Said differently, increasing the density of the struts or filaments 112 may reduce a size of the openings 148. Further, increasing the density of the struts or filaments 112 may increase the quantity of openings 148. The density of the struts or filaments 112 extending across the proximal opening of the lumen 110 may be determined by the viscosity of the fluid that is to pass through the lumen 110. In the present example, the density of the struts or filaments 112 may be selected to allow bile to flow through the lumen 110 from the intrahepatic duct 12 into the stomach 10 while preventing food particles from entering the lumen 110 from the stomach 10. However, the density of the struts or filaments 112 may be determined by the placement location of the stent 100. For example, more viscous fluids or fluids containing particulates may require larger openings 148 (lower density of the struts or filaments 112) while a less viscous or particular free fluid may flow through smaller openings 148 (greater density of the struts 112).The struts or filaments 112 may overlap in any number of patterns or irregularly, as desired. In the illustrated embodiments, the struts or filaments 112 may be arranged as a plurality of loops 150a-l having a generally teardrop shape. While the illustrated embodiment illustrates twelve loops 150a-l, the proximal cage 132 may include fewer than twelve or more than twelve loops 150a-l, as desired. The struts or filaments 112 may be positioned such that a selected loop 150b at least partially overlaps the preceding loop 150a. The overlapping pattern may continue about the circumference of the proximal end 142 such that each loop overlaps preceding loop and said loop is overlapped by the successive loop. It is contemplated that overlapping the loops 150a-l may allow temporary manipulation (e.g., movement of one or more struts or filaments 112) of the proximal cage 132 endoscopically by a clinician, if desired. For example, one or more of the loops 150a-l may be temporarily displaced to allow temporary access of a device to the stent lumen. The device may be larger than what may be allowed by the proximal cage 132 in an unbiased or unmanipulated configuration. In some cases, the struts or filaments 112 may be knit or woven to interlock adjacent loops 150a-l. In other examples, the struts or filaments 112 may be wound such that a successive loop is disposed over a preceding loop. It is contemplated that the loops 150a-l may take other shapes such as, but not limited to, circular, oblong, square, rectangular, diamond shaped, polygonal, irregular, etc. In other examples, the struts or filaments 112 may be woven or otherwise extend across the proximal luminal opening such that the struts or filaments 112 have a generally linear orientation. In some examples, the loops 150a-l (or linearly arranged pattern) may be formed from one continuous strut or filaments 112. In other examples, the loops 150a-l (or linearly arranged pattern) may be formed from two or more struts or filaments 112. The loops 150a-l may extend around an entirety of a circumference of the proximal end 142 of the proximal cage 132 or around an entirety of the proximal luminal opening. In other examples, the loops 150a-l may extend less than 360° around the circumference of the proximal end 142 of the proximal cage 132 or around an entirety of the proximal luminal opening.

The distal end region 134 may include a flare or may gradually increase in outer diameter from a proximal end 152 of the distal end region 134 having an outer diameter similar to or approximately equal to the outer diameter 138 of the intermediate region to a second outer diameter 140 adjacent to or at the distal end 106 of the stent 100. However, this is not required. In some examples, the distal end region 134 may have a same diameter as the diameter 138 of the intermediate region 108 along an entire length of the distal end region 134. In other examples, the distal end region 134 may include an abrupt or stair-step transition between a first outer diameter and the second outer diameter 140.

In some embodiments, the proximal cage 132 may have a first outer diameter 136 and the distal end region 134 may have a second outer diameter 140. The outer diameter 136 of the proximal cage 132 and/or the outer diameter 140 of the distal end region 134 may be greater than the outer diameter 138 of the intermediate region 108. The inner diameter of at least a portion of the proximal cage 132 and/or the distal end region 134 may be greater than the inner diameter of the intermediate region 108. In some instances, the first and second outer diameters 136, 140 may be approximately the same, while in other instances, the first and second outer diameters may be different. For example, in some cases, the outer diameter 136 of the proximal cage 132 may be greater than the outer diameter 140 of the distal end region 134. The stent 100 may take other shapes, as desired. In some embodiments, the distal end region 134 of the stent 100 may include a structure similar to the proximal cage 132. In other embodiments, the stent 100 may have a generally uniform outer diameter from the proximal end 104 to the distal end 106 thereof. It is contemplated that the outer diameter of the stent 100 may be varied to suit the desired application.

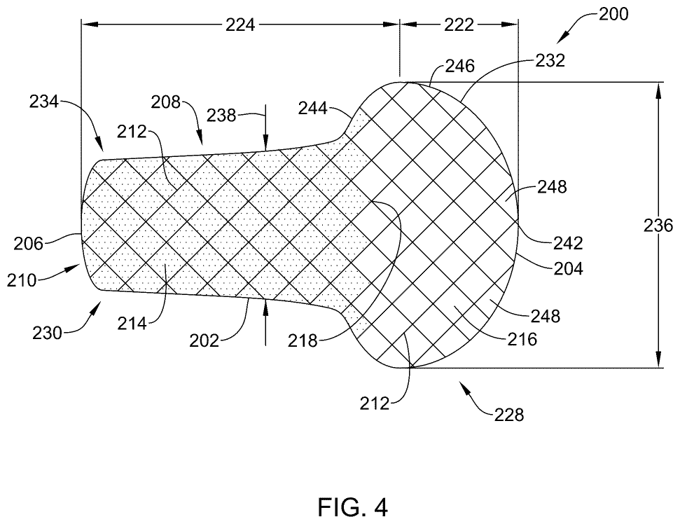

FIG. 4 illustrates a side view of another illustrative implant 200, such as, but not limited to, a stent, in an expanded state. In some instances, the stent 200 may be formed from an elongated tubular member 202. While the stent 200 is described as generally tubular, it is contemplated that the stent 200 may take any cross-sectional shape desired. The stent 200 may have a first, or proximal, end 204, a second, or distal, end 206, and an intermediate region 208 disposed between the first end 204 and the second end 206. The stent 200 may include a lumen 210 extending from a first opening adjacent the first end 204 to a second opening adjacent to the second end 206 to allow for the passage of bile, fluids, and the like therethrough. In some cases, as will be described in more detail herein, the first opening adjacent to the first end 204 may include one or more or a plurality of openings.

The stent 200 may be radially expandable from a first radially collapsed configuration (not explicitly shown) to a second radially expanded configuration, as shown in FIG. 4. The stent 200 may be structured to extend across two non-adherent structures/tissues and to apply a radially outward pressure to create an opening or passage between the two non-adherent structures/tissues, thereby forming an anastomosis between the two separate anatomical structures.

The tubular member 202 of the stent 200 may have a scaffold structure, fabricated from one or more, or a plurality of interwoven filaments or struts 212. The scaffold structure may extend from the first end 204 to the second end 206 of the stent 200. For example, the scaffold structure, and thus the filament(s) thereof, may extend continuously from the first end 204 to the second end 206 of the stent 200. In some embodiments, the stent 200 may be formed with one filament interwoven with itself (e.g., knitted) to form the scaffold structure. In other embodiments, the stent 200 may be formed with several interwoven filaments (e.g., braided) to form the scaffold structure. Thus, in such instances one or more of the filament(s) forming the scaffold structure may extend continuously from the first end 204 to the second end 206 of the stent 200. In still another embodiment, the stent 200 may include a laser cut tubular member to form the scaffold structure. A laser cut tubular member may have an open and/or closed cell geometry including one or more interconnected struts formed as a monolithic structure from the tubular member. In such instances, the laser cut tubular member forming the scaffold structure may extend continuously from the first end 204 to the second end 206 of the stent 200.

In some instances, an inner and/or outer surface of the scaffold structure of the stent 200 may be entirely, substantially or partially, covered with a polymeric covering or layer 214. For example, a covering or coating 214 may extend across the open cells of the scaffold structure to prevent tissue ingrowth into the lumen 210 of the stent 200. In some cases, the covering or coating 214 may prevent food or particulate ingress into the lumen 210 or fluid leakage from the lumen 210 along the coated regions. While not explicitly shown, the covering or coating 214 may include an outer layer disposed over an outer surface of the scaffold structure and/or an inner layer disposed over an inner (e.g., luminal) surface of the scaffold structure. In some embodiments, the stent 200 may include only an outer polymeric covering on an outer surface of the scaffold structure. In other embodiments, the stent 200 may include only an inner polymeric covering on an inner surface of the scaffold structure. In some instances, an inner layer and an outer layer may be formed as a single unitary structure to form the covering or coating 214. In other embodiments, an inner layer and an outer layer may be formed as separate layers to collectively form the covering or coating 214. The inner and outer layers may be formed from the same material or different materials, as desired. The covering or coating 214 may span or be disposed within openings or interstices 216 defined between adjacent stent filaments or struts 212 of the scaffold structure. It can be appreciated that as an inner layer and an outer layer extend outwardly and inwardly, respectively, they may touch and/or form an interface region within the spaces (e.g., openings, cells, interstices) in the wall of the scaffold structure of the stent 200. For example, the inner and outer layers may extend into the openings 216 defined between adjacent stent struts 212 and form an interface region. Further, the inner and outer layers may additionally extend between adjacent filaments or struts 212, thereby filling any space between adjacent filaments or strut members 212, and thus prevent tissue ingrowth into the lumen of the stent 200. The covering or coating 214 may extend along an entire length of the stent 200 from the first end 204 to the second end 206. In other embodiments, the covering or coating 214 may extend along only a portion of the stent 200. For example, the covering or coating 214 may extend distally from a first end 218 distal to the proximal end 204 of the stent 200 to the distal end 206 of the stent 200 to define a proximal end region 222 free from the covering or coating 214 and a coated region 224. In other embodiments, the covering or coating 214 may terminate proximal to the distal end 206 of the stent 200 to define a distal end region free from the covering or coating 214. It is contemplated that the proximal end region 222 may remain uncoated to allow fluid or bile to pass through the lumen 210 of the stent 200 and exit the lumen 210 at the proximal end region 222, while restricting large particle ingress. In some embodiments, the stent 200 may include an uncoated distal end region (not explicitly shown). It is contemplated that the length of the uncoated proximal end region 222, the coated region 224, and/or any other coated or uncoated regions may be determined based on the desired application, implant location, etc. In some examples, the stent 200 may be fully coated with openings for fluid flow processed into portions of the coating 214.

It is contemplated that the scaffold structure, e.g., the filaments and/or struts, of the stent 200 can be made from a number of different materials such as, but not limited to, metals, metal alloys, shape memory alloys, and/or polymers, as desired, enabling the stent 200 to be expanded into shape when accurately positioned within the body. In some instances, the material may be selected to enable the stent 200 to be removed with relative ease as well. For example, the stent 200 can be formed from alloys such as, but not limited to, nitinol and Elgiloy®. Depending on the material selected for construction, the stent 200 may be self-expanding or require an external force to radially expand the stent 200. In some embodiments, filaments may be used to make the stent 200, which may be composite filaments, for example, having an outer shell made of nitinol and having a platinum core. It is further contemplated that the filaments of the stent 200 may be formed from polymers including, but not limited to, polyethylene terephthalate (PET).

In some instances, in the radially expanded configuration, the stent 200 may include a first end region 228 proximate to the first end 204 and a second end region 230 proximate to the second end 206. In some embodiments, the first end region 228 may include a proximal cage 232 positioned adjacent to the first end 204 and the second end region 230 may include a distal end region 234 positioned adjacent to the second end 206 of the stent 200. It is contemplated that the proximal cage 232 and/or the distal end region 234 may be formed at a same time as the elongated tubular member 202 of the stent 200. For example, the proximal cage 232 and/or the distal end region 234 may be formed as the elongated tubular member 202 is woven, knit, laser cut, or the like. The proximal cage 232 may be configured to engage an interior portion of the wall of the body cavity or body lumen. For example, the proximal cage 232 may be positioned against an interior of a first body lumen (e.g., the wall of the stomach 10). The distal end region 234 may be configured to be positioned within another separate lumen (e.g., duct 12). However, while not explicitly shown, the distal end region 234 may increase in diameter relative to the intermediate region 208 to exert a radially outward force on a surface of the lumen to facilitate anchoring of the stent 200, similar to the distal end region 134 shown and described with respect to FIG. 2. The stent 200 may be positioned to traverse between two separate anatomical structures. In one example, the proximal cage 232 may be positioned in the stomach 10 and the distal end region 234 may be positioned in the intrahepatic duct 12. The intermediate region 208 of the stent 200 extending between the proximal cage 232 and the distal end region 234 may extend through the wall of the stomach 10 and into the lumen of the intrahepatic duct 12, interconnecting the two anatomical structures. In some cases, at least a portion of the proximal cage 232 may be configured to contact an interior of the wall of the stomach 10 and/or the distal end region 234 may contact an inner surface of the intrahepatic duct 12. The proximal cage 232 and/or the distal end region 234 may anchor the stent 200 against the stomach wall and the intrahepatic duct 12 so that stent 200 is inhibited or prevented from migrating. However, this is not required.

The proximal cage 232 may have an outer diameter 236 that is greater than an outer diameter 238 of the intermediate region 208. In some examples, the proximal cage 232 may have a generally hemi-spherical shape including a proximal end 242, a distal shoulder 244, and a curved side wall 246 extending between the proximal end 242 and the distal shoulder 244. It is contemplated that the proximal cage 232 may take other shapes, such as, but not limited to, spherical, spheroid, rectangular prism, irregular, or the like. The hemi-spherical shape of the proximal cage 232 may increase the surface area through which fluid can exit the lumen 210 of the stent 200 (e.g., flowing in the distal to proximal direction) while still restricting the flow of food particles in the reverse direction (e.g., in the proximal to distal direction), relative to a spheroidal shape. For example, increasing the surface area of the proximal cage 232 may increase the surface area of the openings 248 available for fluid to pass through when compared to a proximal cage having a smaller surface area and same size openings. Said differently, increasing the overall surface area of the proximal cage 232 may increase a number of openings 248 available for fluid to flow through while maintaining a same size opening (relative to a proximal cage having a smaller overall surface area).

The distal shoulder 244 of the proximal cage 232 may extend radially from the intermediate region 208 of the elongated tubular member 202. It is contemplated that the transition from the cross-sectional area of the intermediate region 208 to the proximal cage 232 may be gradual, sloped, or occur in an abrupt step-wise manner, as desired. In some examples, the distal shoulder 244 may extend generally orthogonal to a longitudinal axis of the elongated tubular member 202. However, it is contemplated that the distal shoulder 244 may extend at non-orthogonal angles, as desired. The angle of the distal shoulder 244 may be selected based on the target anatomy. In some cases, the distal shoulder 244 may provide a mechanical stop between the proximal cage 232 and the target anatomy. The curved side wall 246 may have a curved shape that gradually increases in cross-sectional dimension and then gradually decreases in cross-sectional dimension in a direction parallel to the longitudinal axis of the elongated tubular body 202. However, the curved side wall 246 may take other shapes including curved, linear, or irregular shapes, as desired.

The curved side wall 246 and/or the proximal end 242 of the proximal cage 232 may include more struts 212 of the elongated body 202 extending across the proximal opening of the lumen 210 of the stent 200. Said differently, the struts 212 may bisect the proximal luminal opening of the stent 200 to form a mesh or screen. The struts 212 may extend transverse to a longitudinal axis of the stent 200. The one or more struts 212 may intersect or overlap with one another to define a plurality of openings 248 each having a smaller cross-sectional dimension than the cross-sectional dimension of the elongated tubular member 202 or the transluminal opening. For purposes of clarity and simplicity, not every opening 248 is labeled. The openings 248 may be similar to openings 216. At least some of the openings 248 of the proximal cage 232 may be disposed radially inward of the intermediate region 208 of the stent 200. It is contemplated that the density or number of the struts 212 extending across the proximal opening may be inversely proportional to the size of the openings 248. For example, the denser the struts 212 (e.g., the greater the number of struts 212 or the greater the number of intersections or overlaps), the smaller the openings 248. Said differently, increasing the density of the struts 212 may reduce a size of the openings 248. Further, increasing the density of the struts 212 may increase the quantity of openings 248. The density of the struts 212 extending across the proximal opening of the lumen 210 may be determined by the viscosity of the fluid that is to pass through the lumen 210. In the present example, the density of the struts 212 may be selected to allow bile to flow through the lumen 210 from the intrahepatic duct 12 into the stomach 10 while preventing food particles from entering the lumen 210 from the stomach 10. However, the density of the struts 212 may be determined by the placement location of the stent 200.

The struts 212 may overlap in any number of patterns or irregularly, as desired. In the illustrated embodiments, the struts 212 may be arranged in a similar manner to the intermediate region 208 of the stent 200. For example, the struts 212 of the proximal cage 232 may be interwoven (or laser cut) into a scaffold structure. In other examples, the proximal cage 232 may include a plurality of loops or a wound structure similar to the loops 150a-l of FIG. 3. The openings 248 of the proximal cage 232 may take any shape desired, such as, but not limited to circular, oblong, square, rectangular, diamond shaped, polygonal, irregular, etc. In other examples, the struts 212 may be woven or otherwise extend across the proximal luminal opening such that the struts 212 have a generally linear orientation. It is contemplated that the struts 212 of the proximal cage 132 may be temporarily manipulated (e.g., movement of one or more struts 212) endoscopically by a clinician, if desired. For example, one or more of the struts 212 may be temporarily displaced to allow temporary access of a device to the stent lumen. The device may be larger than what may be allowed by the proximal cage 232 in an unbiased or unmanipulated configuration.

The distal end region 234 may have an outer diameter similar to or approximately equal to the outer diameter 238 of the intermediate region 208. However, this is not required. In some embodiments, the distal end region may include a flare or may gradually increase in outer diameter from a proximal end of the distal end region 234 having an outer diameter similar to or approximately equal to the outer diameter 238 of the intermediate region to a second outer diameter adjacent to or at the distal end 206 of the stent 200. In other examples, the distal end region 234 may include an abrupt or stair-step transition between a first outer diameter and the second outer diameter.

In some embodiments, the outer diameter 236 of the proximal cage 232 and/or the outer diameter of the distal end region 234 may be greater than the outer diameter 238 of the intermediate region 208. The inner diameter of at least a portion of the proximal cage 232 and/or the distal end region 234 may be greater than the inner diameter of the intermediate region 208. In some instances, the first and second outer diameters of the proximal cage 232 and the distal end region 234 may be approximately the same, while in other instances, the first and second outer diameters may be different. For example, in some cases, the outer diameter 236 of the proximal cage 232 may be greater than the outer diameter of the distal end region 234. The stent 200 may take other shapes, as desired. In some embodiments, the distal end region 234 of the stent 200 may include a structure similar to the proximal cage 232. In other embodiments, the stent 200 may have a generally uniform outer diameter from the proximal end 204 to the distal end 206 thereof. It is contemplated that the outer diameter of the stent 200 may be varied to suit the desired application.

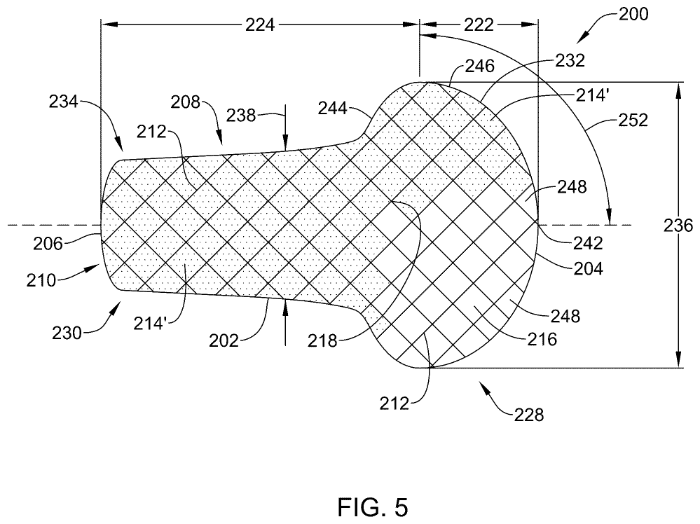

FIG. 5 illustrates a side view of the illustrative stent 200 of FIG. 4 having an alternative coating 214′ arrangement. In the illustrated embodiment of FIG. 5, the coating 214′ extends proximally to the proximal end 242 of the proximal cage 232 over a portion thereof. For example, the coating 214′ may extend radially over a portion 252 of the proximal cage 232. In some examples, the stent 200 may be deployed within the body such that the coated portion 252 of the proximal cage 232 is positioned to shield the lumen 210 of the stent 200 from food as it exits the esophagus. The stent delivery system may include visual indicia to help the clinician position the stent 200 with the coated portion 252 of the proximal cage 232 in the desired orientation relative to the patient's anatomy. In some examples, the coating 214′ may be applied to selectively drain the lumen 210 in a desired direction. It is contemplated that one or more regions of the stent 200 may remain free from the coating 214′ to control a flow of fluid. The one or more regions of the stent 200 free from the coating 214′ may be positioned anywhere along a length of the stent 200 and/or circumference thereof and/or anywhere at the proximal cage 232 desired to control the flow of fluid and/or limit or prevent particle ingress. As shown in FIG. 5, a first portion of the proximal cage 232 may be covered with the coating ′214 while a second portion f the proximal cage 232 may remain uncovered, allowing fluid to flow through interstices of the uncovered portion of the proximal cage 232. When implanted, the covered portion of the proximal cage 232 may be oriented toward the lower end of the esophagus, shielding the proximal cage 232 from particle (e.g., food, fluid, etc.) ingress into the cage from the esophagus.

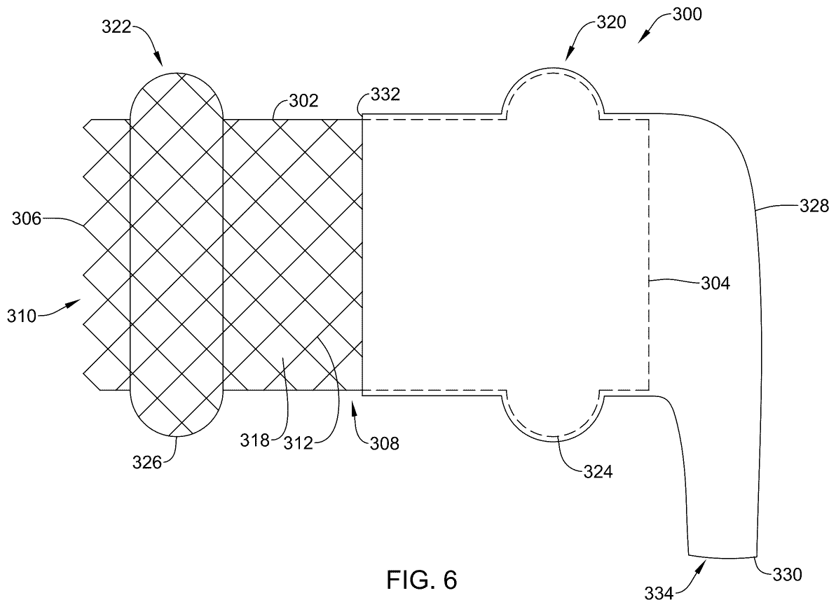

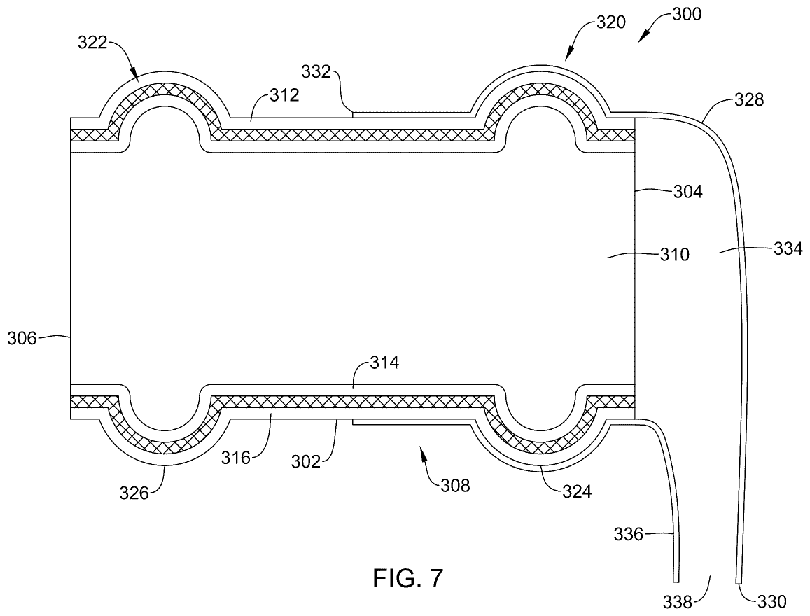

FIG. 6 illustrates a side view of another illustrative implant 300, such as, but not limited to, a stent, in an expanded state. FIG. 7 illustrates a cross-sectional view of the illustrative stent 300 of FIG. 6. In some instances, the stent 300 may be formed from an elongated tubular member 302. While the stent 300 is described as generally tubular, it is contemplated that the stent 300 may take any cross-sectional shape desired. The stent 300 may have a first, or proximal, end 304, a second, or distal, end 306, and an intermediate region 308 disposed between the first end 304 and the second end 306. In some cases, the intermediate region 308 may be referred to as a saddle region. The stent 300 may include a lumen 310 extending from a first opening adjacent the first end 304 to a second opening adjacent to the second end 306 to allow for the passage of bile, fluids, and the like therethrough.

The stent 300 may be radially expandable from a first radially collapsed configuration (not explicitly shown) to a second radially expanded configuration, as shown in FIGS. 6-7. The stent 300 may be structured to extend across two non-adherent structures/tissues and to apply a radially outward pressure to create an opening or passage between the two non-adherent structures/tissues, thereby forming an anastomosis between the two separate anatomical structures.

The tubular member 302 of the stent 300 may have a scaffold structure, fabricated from one or more, or a plurality of interwoven filaments or struts 312. The scaffold structure may extend from the first end 304 to the second end 306 of the stent 300. For example, the scaffold structure, and thus the filament(s) thereof, may extend continuously from the first end 304 to the second end 306 of the stent 300. In some embodiments, the stent 300 may be formed with one filament interwoven with itself (e.g., knitted) to form the scaffold structure. In other embodiments, the stent 300 may be formed with several interwoven filaments (e.g., braided) to form the scaffold structure. Thus, in such instances one or more of the filament(s) forming the scaffold structure may extend continuously from the first end 304 to the second end 306 of the stent 300. In still another embodiment, the stent 300 may include a laser cut tubular member to form the scaffold structure. A laser cut tubular member may have an open and/or closed cell geometry including one or more interconnected struts formed as a monolithic structure from the tubular member. In such instances, the laser cut tubular member forming the scaffold structure may extend continuously from the first end 304 to the second end 306 of the stent 300.

In some instances, an inner and/or outer surface of the scaffold structure of the stent 300 may be entirely, substantially or partially, covered with a polymeric covering or layer 314, 316 (see, for example, FIG. 7). For example, a covering or coating may extend across the open cells of the scaffold structure to prevent tissue ingrowth into the lumen of the stent 300. In some cases, the covering or coating 314, 316 may prevent food or particulate ingress into the lumen 310 or fluid leakage from the lumen 310 along the coated regions. However, in some embodiments one or both of the polymeric coverings 314, 316 may be omitted. For example, in some embodiments the stent 300 may include only the outer polymeric covering 316 on an outer surface of the scaffold structure. In other embodiments, the stent 300 may include only the inner polymeric covering 314 on an inner surface of the scaffold structure. In some instances, the inner layer 314 and the outer layer 316 may be formed as a single unitary structure. In other embodiments, the inner layer 314 and the outer layer 316 may be formed as separate layers. The inner and outer layers 314, 316 may be formed from the same material or different materials, as desired. The inner layer 314 and/or outer layer 316 may span or be disposed within openings or interstices defined between adjacent stent filaments or struts 312 of the scaffold structure. It can be appreciated that as inner layer 314 and outer layer 316 extend outwardly and inwardly, respectively, they may touch and/or form an interface region within the spaces (e.g., openings, cells, interstices) 318 in the wall of the scaffold structure of the stent 300. For example, the inner and outer layers 314, 316 may extend into the openings 318 defined between adjacent stent struts 312 and form an interface region. Further, the inner and outer layers 314, 316 may additionally extend between adjacent filaments or struts 312, thereby filling any space between adjacent filaments or strut members 312, and thus prevent tissue ingrowth into the lumen of the stent 300.

It is contemplated that the scaffold structure, e.g., the filaments and/or struts, of the stent 300 can be made from a number of different materials such as, but not limited to, metals, metal alloys, shape memory alloys, and/or polymers, as desired, enabling the stent 300 to be expanded into shape when accurately positioned within the body. In some instances, the material may be selected to enable the stent 300 to be removed with relative ease as well. For example, the stent 300 can be formed from alloys such as, but not limited to, nitinol and Elgiloy®. Depending on the material selected for construction, the stent 300 may be self-expanding or require an external force to radially expand the stent 300. In some embodiments, filaments may be used to make the stent 300, which may be composite filaments, for example, having an outer shell made of nitinol and having a platinum core. It is further contemplated that the filaments of the stent 300 may be formed from polymers including, but not limited to, polyethylene terephthalate (PET).

In some instances, in the radially expanded configuration, the stent 300 may include a first end region 320 proximate to the first end 304 and a second end region 322 proximate to the second end 306. In some embodiments, the first end region 320 and the second end region 322 may include shoulders or enlarged regions, such as flanges 324, 326 positioned adjacent to the first end 304 and the second end 306 of the stent 300. The flanges 324, 326 may be configured to engage an interior portion of the walls of the body cavity or body lumen. For example, the first flange 324 may be positioned against an interior of a first body lumen and the second flange 326 may be positioned against an interior of a second body lumen different from the first body lumen. Thus, the stent 300 may be positioned to traverse between two separate anatomical structures. The first and/or second flanges 324, 326 may anchor the stent 300 against the anatomical structures so that the stent 300 is inhibited or prevented from migrating. However, this is not required.

The flanges 324, 326 may extend circumferentially around the stent 300, and define an annular pocket extending circumferentially around an interior of the stent 300. The annular pocket may have an enlarged inner diameter relative to portions of the flange 324, 326 on either side thereof. In some embodiments, the flanges 324, 326 may have a larger diameter than the intermediate region or saddle 308 of the stent 300 located between the end regions 320, 322 to prevent or help prevent the stent 300 from migrating once placed within a body cavity, body lumen, or across body cavities or lumens. It is contemplated that the transition from the cross-sectional area of the intermediate region or saddle 308 to the retention features or flanges 324, 326 may be gradual, sloped, or occur in an abrupt step-wise manner, as desired. In some cases, the flanges 324, 326 may have a curved semi-circular cross-sectional shape that gradually increases in cross-sectional dimensions and then gradually decreases in cross-sectional dimension in a direction such that the first and/or second ends 304, 306 have a similar cross-sectional dimension to the intermediate region or saddle 308. However, this is not required. Other shapes and/or configurations may be used, as desired. For example, in some cases, a distal side of the proximal flange 324 and a proximal side of the distal flange 326 may have a generally concave shape. In some examples, one or both of the flanges 324, 326 may be omitted. In some embodiments, the stent 300 may be similar in form and function to the stents 100, 200, described herein. For example, the stent 300 may include a proximal cage including struts bisecting the proximal luminal opening and/or flared distal end region.

In some embodiments, the first flange 324 may have a first outer diameter and the second flange 326 may have a second outer diameter. The outer diameter of the first flange 324 and/or the second flange 326 may be greater than the outer diameter of the intermediate region or saddle 308. The inner diameter of the first flange 324 and/or the second flange 326 may be greater than the inner diameter of the intermediate region or saddle 308. In some instances, the first flange 324 may have an inner diameter greater than the inner diameter at the first end 304 of the stent 300 and/or the second flange 326 may have an inner diameter greater that the inner diameter at the second end 306 of the stent 300. Thus, the first flange 324 may have a greater inner diameter than portions of the stent 300 extending in opposite directions from the first flange 324 and/or the second flange 326 may have a greater inner diameter than portions of the stent 300 extending in opposite directions from the second flange 326. In some instances, the first and second outer diameters may be approximately the same, while in other instances, the first and second outer diameters may be different. In some embodiments, the stent 300 may include only one flange 324, 326, or the stent 300 may not include a flange, if desired. For example, the first end region 320 may include a flange 324 while the second end region 322 may have an outer diameter similar to that of the intermediate region or saddle 308. It is further contemplated that the second end region 322 may include a flange 326 while the first end region 320 may have an outer diameter similar to that of an outer diameter of the intermediate region or saddle 308. In some embodiments, the stent 300 may have a uniform outer diameter from the first end 304 to the second end 306. It is contemplated that the outer diameter of the stent 300 may be varied to suit the desired application.

The stent 300 may further include a flexible sleeve 328 extending from a proximal end 330 to a distal end 332. The flexible sleeve 328 may be formed from highly flexible polymers that are strong in thin sections, such as, but not limited to, polytetrafluoroethylene (PTFE), expanded polytetrafluoroethylene (ePTFE), low density polyethylene (LDPE), very low density polyethylene (VLDPE), linear low density polyethylene (LLDPE), polyvinyl chloride (PVC), silicone, or the like. The proximal end 330 of the flexible sleeve 328 may extend proximally to the proximal end 304 of the stent 300. In some cases, the distal end 332 of the flexible sleeve 328 may be positioned proximal to the distal end 306 of the stent 300. However, this is not required. In other embodiments, the distal end 332 of the flexible sleeve 328 may extend to the distal end 306 of the stent 300. The flexible sleeve 328 may have a generally tubular configuration defining a lumen 334 extending from the proximal end 330 to the distal end 332 thereof. The lumen 334 of the flexible sleeve 328 is in fluid communication with the lumen 310 of the stent 300.

The flexible sleeve 328 may have a flexibility such that in the absence of a support structure, the wall 336 of the flexible sleeve 328 drapes over the proximal opening of the stent 300. Said differently, the proximal end 330 of the flexible sleeve 328 may be droop below the longitudinal axis of the stent 300. In some embodiments, the flexible sleeve 328 may collapse flat onto itself unless an inner fluid is weeping and/or pushing out of the lumen 310 of the stent 300 into the lumen 334 of the flexible sleeve. It is contemplated that the flexible sleeve 328 may selectively block the proximal opening of the stent 300. For example, fluid may easily pass through the lumen 310 of the stent 300 in a distal to proximal direction, exiting the flexible sleeve 328 at a proximal opening 338 thereof. However, in the example of a stent 300 implanted extending between the stomach 10 and intrahepatic duct 12, food may not be able to enter the proximal opening 338 of the flexible sleeve 328. For example, external forces in the stomach (e.g., food or fluid) may not be able to open the flexible sleeve 328 and thus food or fluid from the stomach may not flow backwards to enter the proximal opening 338 of the flexible sleeve 328. As such, the flexible sleeve 328 may function as a one-way valve controlling the direction flow of substances through the lumen 310 of the stent 300.

In some embodiments, the flexible sleeve 328 may be secured to an outer surface of the stent 300, as shown in FIG. 7. It is contemplated that the flexible sleeve 328 may be coupled to the outer polymeric coating 316 using suitable techniques such as thermal bonding, adhesives, or the like. In other examples, the flexible sleeve 328 may be formed as a single monolithic structure with the outer polymeric coating 316.

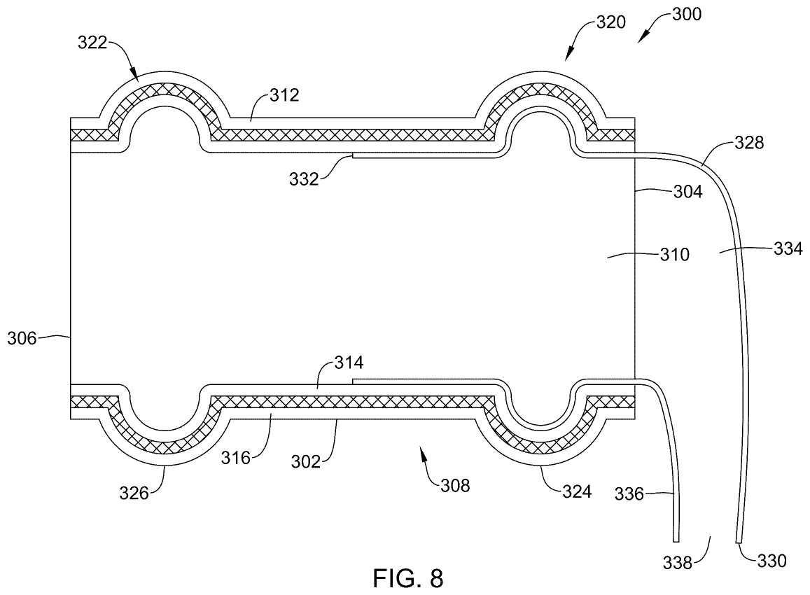

Referring briefly to FIG. 8, which is a cross-sectional view of the illustrative stent 300 having an alternative placement of the flexible sleeve 328, the flexible sleeve 328 may be disposed within the lumen 310 of the stent and coupled to the inner polymeric coating 314. It is contemplated that the flexible sleeve 328 may be coupled to the inner polymeric coating 314 using suitable techniques such as thermal bonding, adhesives, or the like. In other examples, the flexible sleeve 328 may be formed as a single monolithic structure with the inner polymeric coating 314. While FIG. 8 illustrates the flexible sleeve 328 as conforming to the inner surface of the stent 300, in some embodiments, the flexible sleeve 328 may not extend into the flange 324. Said differently, the flexible sleeve 328 may have a uniform or substantially constant inner diameter.

In some examples, whether the flexible sleeve 328 is secured relative to the outer surface of the stent 300 or the inner surface of the stent 300, the inner diameter of the flexible sleeve 328 may reduce in the proximal direction. It is contemplated that reducing the inner diameter of the flexible sleeve 328 may increase the valve-like properties of flexible sleeve 328. For example, fluid may pass easily through the lumen 310 of the stent 300 and into the lumen 334 of the sleeve 328 in the distal to proximal direction while a smaller proximal opening 338 (relative to the proximal opening of the stent 300) may further inhibit flow of food, particles, fluid, or the like in the proximal to distal direction.

The materials that can be used for the various components of the medical stent(s) and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent, the filaments, the covering, flexible sleeve, and/or elements or components thereof.

In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A transluminal implant, comprising:
an elongated tubular body extending from a first end to a second end and comprising:
a scaffolding forming a plurality of cells and defining a lumen extending from the first end to the second end of the elongated tubular body;
a proximal cage disposed adjacent to the first end of the elongated tubular body; and
at least one strut forming a proximal portion of the proximal cage and bisecting a luminal opening of the scaffolding to define two or more openings each having a cross-sectional dimension less than a cross-sectional dimension of the luminal opening.

2. The transluminal implant of claim 1, wherein the proximal cage comprises a distal shoulder, a proximal end, and a curved side wall extending between the distal shoulder and the proximal end.

3. The transluminal implant of claim 2, wherein the distal shoulder extends radially from an intermediate region of the elongated tubular body.

4. The transluminal implant of claim 1, wherein the proximal cage has a generally oblate spheroid shape.

5. The transluminal implant of claim 1, wherein the proximal cage has a generally hemi-spherical shape.

6. The transluminal implant of claim 1, wherein the proximal cage has an outer diameter greater than an outer diameter of the second end of the elongated tubular body.

7. The transluminal implant of claim 1, wherein the at least one strut forms a plurality of loops.

8. The transluminal implant of claim 7, wherein a loop of the plurality of loops at least partially overlaps a preceding loop of the plurality of loops.

9. The transluminal implant of claim 7, wherein the plurality of loops extend about an entirety of a circumference of the luminal opening.

10. The transluminal implant of claim 1, wherein the at least one strut intersects with another strut across the luminal opening.

11. The transluminal implant of claim 1, further comprising a coating disposed over at least some of the plurality of cells.

12. The transluminal implant of claim 11, wherein the two or more openings are free from the coating.

13. The transluminal implant of claim 11, wherein the coating is disposed over at least some of the two or more openings.

14. A transluminal implant, comprising:
an elongated tubular body extending from a proximal end to a distal end and comprising:
a scaffolding forming a plurality of cells and defining a lumen extending from the proximal end to the distal end of the elongated tubular body;
a proximal cage disposed adjacent to the proximal end of the elongated tubular body, the proximal cage including a mesh bisecting a luminal opening of the scaffolding to define a plurality of openings each having a cross-sectional dimension less than a cross-sectional dimension of the luminal opening; and
a coating disposed over a portion of the scaffolding.

15. The transluminal implant of claim 14, wherein at least some of the plurality of openings are free from the coating.

16. The transluminal implant of claim 14, wherein a distal end region of the elongated tubular body is free from the coating.

17. The transluminal implant of claim 14, wherein the mesh comprises one or more intersecting struts.

18. The transluminal implant of claim 14, wherein the mesh comprises a plurality of overlapping loops.

19. A transluminal implant, comprising:
an elongated tubular body extending from a first end to a second end and comprising:
a scaffolding forming a plurality of cells and defining a lumen extending from the first end to the second end of the elongated tubular body;
a coating disposed over at least some of the plurality of cells; and
a flexible sheath extending from a proximal end to a distal end and defining a lumen extending therethrough, the proximal end extending proximally beyond the first end of the elongated tubular body.

20. The transluminal implant of claim 19, wherein an inner diameter of the flexible sheath reduces towards the proximal end of the flexible sheath.
